(19) United States
(12) Patent Application Publication
(10) Pub. No.: US 2026/0249071 A1
(43) Pub. Date: Aug. 27, 2026

(54) DRIVE ASSEMBLY WITH FLUID SEAL FOR USE WITH A BLOOD PUMP

(71) Applicant: BOSTON SCIENTIFIC SCIMED, INC., Maple Grove, MN (US)

(72) Inventors: Tim O'Connor, Claregalway (IE); Saeed Bahrami, Galway (IE); Chris Cullen, Galway (IE); Richard O'Sullivan, Turloughmore (IE)

(73) Assignee: BOSTON SCIENTIFIC SCIMED, INC., MAPLE GROVE, MN (US)

(21) Appl. No.: 19/549,255

(22) Filed: Feb. 25, 2026

Related U.S. Application Data
(60) Provisional application No. 63/763,527, filed on Feb. 26, 2025.

(51) Int. Cl. A61M 60/827; A61M 60/117; A61M 60/226; A61M 60/416

(52) U.S. Cl. CPC A61M 60/827; A61M 60/117; A61M 60/226; A61M 60/416

(57) ABSTRACT

Disclosed are percutaneous circulatory support devices and related blood pump systems that may include an impeller housing including an impeller assembly which includes an impeller rotatably positioned within the impeller housing. A motor may be positioned proximal of the impeller assembly and a drive shaft may be rotationally driven by the motor. A drive sleeve may be disposed circumferentially around at least a portion of the drive shaft. A seal assembly including a seal housing and a sealing member may be secured to the seal housing, whereby the seal housing is fixed relative to the impeller housing. The sealing member may contact a circumferential surface of the drive sleeve in sealing engagement therewith such that the drive shaft is rotatable with the impeller.

### CROSS-REFERENCE TO RELATED APPLICATION

This application claims the benefit of U.S. Patent Application Serial No. 63/763,527, filed February 26, 2025, entitled " DRIVE ASSEMBLY WITH FLUID SEAL FOR USE WITH A BLOOD PUMP”, which is incorporated by reference herein in its entirety.

### TECHNICAL FIELD

The present disclosure pertains to percutaneous blood pumps and related devices. More particularly, the present disclosure pertains to sealing assemblies for use with percutaneous blood pumps and associated drive mechanisms, systems, designs, components, processes and the like.

### BACKGROUND

Percutaneous mechanical circulatory support devices, such as blood pumps can provide transient support in patients whose heart function or cardiac output is compromised. The percutaneous mechanical circulatory support devices may be sufficiently flexible to be navigated through the vasculature to a patient’s heart. Such devices may be navigated through the aortic arch and placed across the aortic valve into the ventricle, for example. Various configurations of percutaneous mechanical circulatory support devices are known. However, there is an ongoing need to provide improved construction of percutaneous mechanical circulatory support devices.

### BRIEF SUMMARY

This disclosure provides design, material, manufacturing method, and use alternatives for medical devices, including percutaneous circulatory support devices and associated blood pump and drive devices.

A first example is a percutaneous blood pump including an impeller housing. An impeller assembly may be disposed within the impeller housing, the impeller assembly including an impeller rotatably positioned within the impeller housing. A motor may be positioned proximal of the impeller assembly whereby a drive shaft is rotationally driven by the motor. A seal assembly including a seal housing and a sealing member may be secured to the seal housing. The seal assembly, such as the seal housing, may be fixed relative to the impeller housing, whereby the sealing member contacts a circumferential surface of the drive sleeve in sealing engagement therewith such that the drive shaft is rotatable with the impeller.

Alternatively or additionally to any of the examples described herein, the seal assembly includes a spring member configured to apply a force to the sealing member to urge the sealing member against the circumferential surface of the drive sleeve.

Alternatively or additionally to any of the examples described herein, the spring member may store potential energy and discharge kinetic energy in a radially inward direction to maintain sealing engagement between the sealing member and the drive sleeve.

Alternatively or additionally to any of the examples described herein, sealing engagement between the sealing member and the drive sleeve may be maintained as the drive shaft rotates.

Alternatively or additionally to any of the examples described herein, sealing engagement between the sealing member and the drive sleeve may be maintained by the spring member as the drive shaft rotates.

Alternatively or additionally to any of the examples described herein, the drive sleeve may be composed of stainless steel and/or polished stainless steel.

Alternatively or additionally to any of the examples described herein, the drive sleeve has a surface roughness of Ra 0.8 µm or less.

Alternatively or additionally to any of the examples described herein, the drive shaft may extend distally from the motor into the impeller housing.

Alternatively or additionally to any of the examples described herein, the drive sleeve is fixed to the impeller assembly and rotatable therewith.

Alternatively or additionally to any of the examples described herein, the drive sleeve is fixed to a distal end region of the drive shaft.

Alternatively or additionally to any of the examples described herein, whereby a distance from a distal end of the motor to a proximal end of the impeller is 10 mm or less.

Another example is a percutaneous blood pump including an impeller housing. An impeller assembly may be disposed within the impeller housing, the impeller assembly including an impeller rotatably positioned within the impeller housing. A motor may be positioned proximal of the impeller assembly and including a drive shaft extending distally therefrom, the motor configured to rotationally drive the impeller assembly. A drive sleeve may circumferentially surround and be affixed to a distal end portion of the drive shaft. A seal assembly including a seal housing and a sealing member may be secured to the seal housing, whereby the sealing member contacts a circumferential surface of the drive sleeve in sealing engagement therewith such that the drive sleeve is rotatable therein with rotation of the impeller.

Alternatively or additionally to any of the examples described herein, the seal assembly may include a spring member configured to apply a force to the sealing member to urge the sealing member against the circumferential surface of the drive sleeve.

Alternatively or additionally to any of the examples described herein, the spring member may store potential energy and discharge kinetic energy in a radially inward direction to maintain sealing engagement between the sealing member and the drive sleeve.

Alternatively or additionally to any of the examples described herein, whereby sealing engagement between the sealing member and the drive sleeve is maintained as the drive shaft rotates.

Alternatively or additionally to any of the examples described herein, the drive sleeve has a surface roughness of Ra 0.8 µm or less.

Another example is a percutaneous blood pump including an impeller housing. A motor housing may be positioned proximal of the impeller housing. An impeller assembly may be disposed within the impeller housing, the impeller assembly including an impeller rotatably positioned within the impeller housing. A motor may be disposed within the motor housing. A drive shaft may be rotationally driven by the motor, including a drive sleeve circumferentially surrounding a distal end region of the drive shaft and affixed thereto. The drive sleeve may be operably coupled to the impeller assembly such that the drive sleeve rotates with the drive shaft and the impeller. A seal assembly may be disposed within the impeller housing, the seal assembly including a lip seal circumferentially surrounding the drive sleeve and forming a fluid seal therebetween. Whereby the lip seal may provide a fluid seal between the impeller housing and the motor housing.

Alternatively or additionally to any of the examples described herein, the lip seal may be a spring-loaded, self-energizing lip seal.

Alternatively or additionally to any of the examples described herein, the drive sleeve includes a distal end region having a first diameter and a proximal end region having a second diameter less than the first diameter.

Alternatively or additionally to any of the examples described herein, the lip seal surrounds and contacts the proximal end region of the drive sleeve proximal of the distal end region.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump;

FIG. 2 shows the distal end region of the percutaneous circulatory support device of FIG. 1 including the percutaneous blood pump;

FIG. 3 is a side view of a portion of the percutaneous blood pump of FIG. 1;

FIG. 4 is a cross-sectional view of the portion of the percutaneous blood pump of FIG. 3;

FIG. 5 is an enlarged view of an example sealing drive mechanism of the percutaneous circulatory support device of FIG. 4;

FIG. 6A is perspective cross-sectional view of a seal assembly in accordance with the present disclosure;

FIG. 6B is a schematic cross-sectional view of the seal assembly shown in FIG. 6A and in accordance with the present disclosure; and

FIG. 7 is a schematic cross-sectional view of an alternative seal assembly in accordance with the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

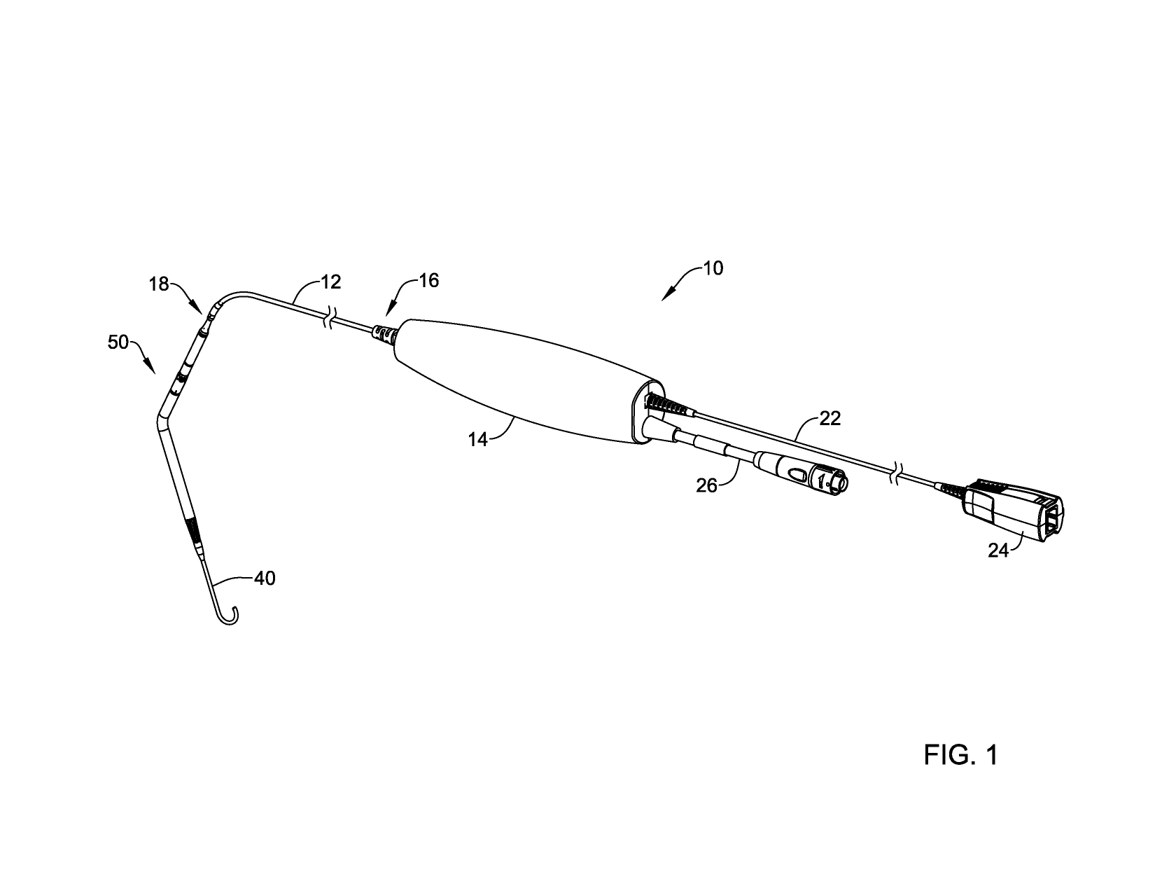

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. A proximal end 16 of the elongate shaft 12 may be coupled to a junction housing 14 and a distal end 18 of the elongate shaft 12 may be coupled to the percutaneous blood pump 50. An electrical cable 22 may extend from the junction housing 14 to a connector 24 at a proximal end thereof. The connector 24 may be configured to be connected to a controller (not shown) for controlling the blood pump 50, such as providing electrical power to the blood pump 50. The percutaneous circulatory support device 10 may also include an extension 26 connectable to the controller for sending and/or receiving signals, such as from one or more sensors during operation of the blood pump 50.

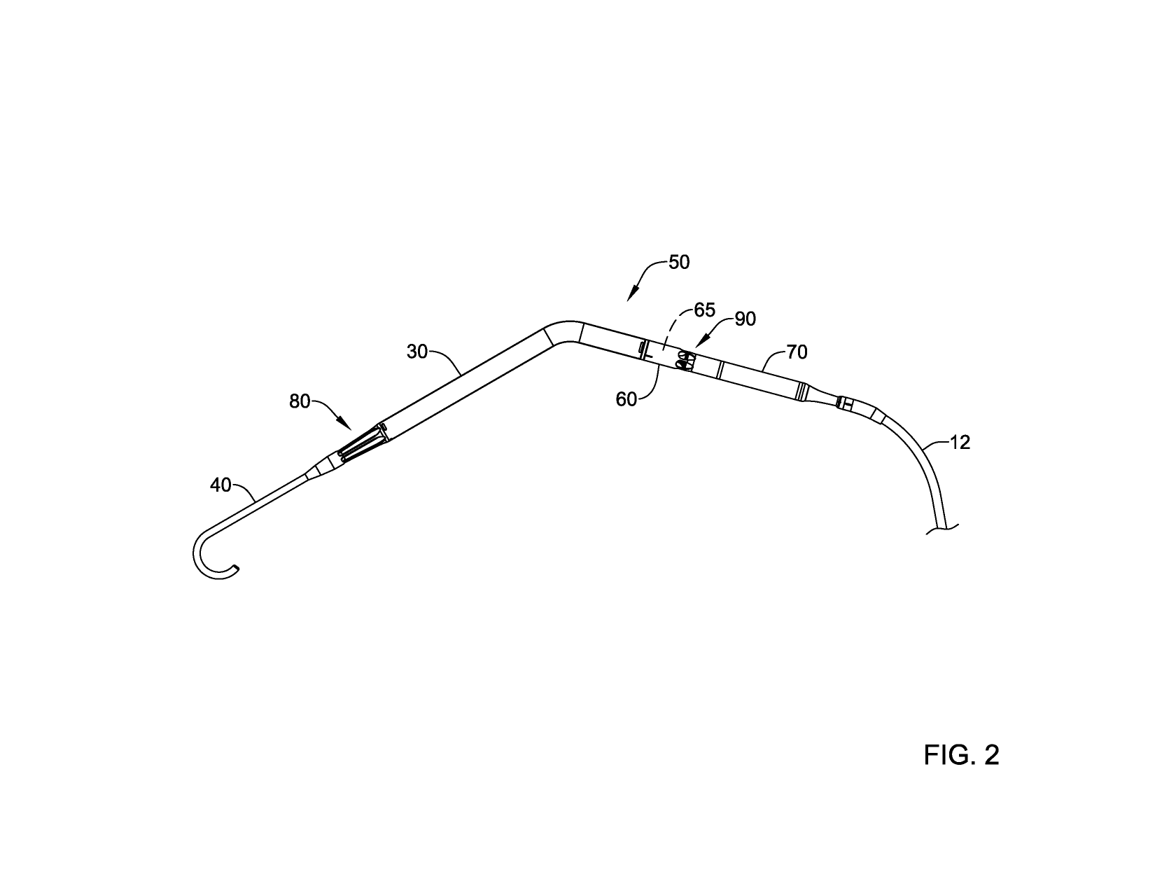

Additional features of the blood pump 50 are illustrated in FIG. 2 which shows the distal end region of the percutaneous circulatory support device of FIG. 1 including the percutaneous blood pump. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60 and/or the motor housing 70 may be separate components. The impeller housing 60 carries an impeller assembly 65 therein. The impeller assembly 65 may include an impeller, secured to an impeller shaft, that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on, within, or proximate other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller, such as an impeller shaft of the impeller, may be directly or indirectly coupled to the drive shaft of the motor such that rotation of the drive shaft of the motor directly rotates the impeller. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with a driven magnet. In other embodiments, the motor may be coupled to the impeller assembly 65 via other components.

The blood pump 50 may be guided over a guidewire during introduction of the blood pump 50 into the vasculature of a patient. For instance, a guidewire, inserted through a guidewire lumen of the distal tip 40, may be advanced proximally along the impeller assembly 65 and out through one of the outflow windows of the blood outlet 90. With the guidewire tracked through the blood pump 50, the percutaneous circulatory support device 10 may be advanced over the guidewire into a vasculature.

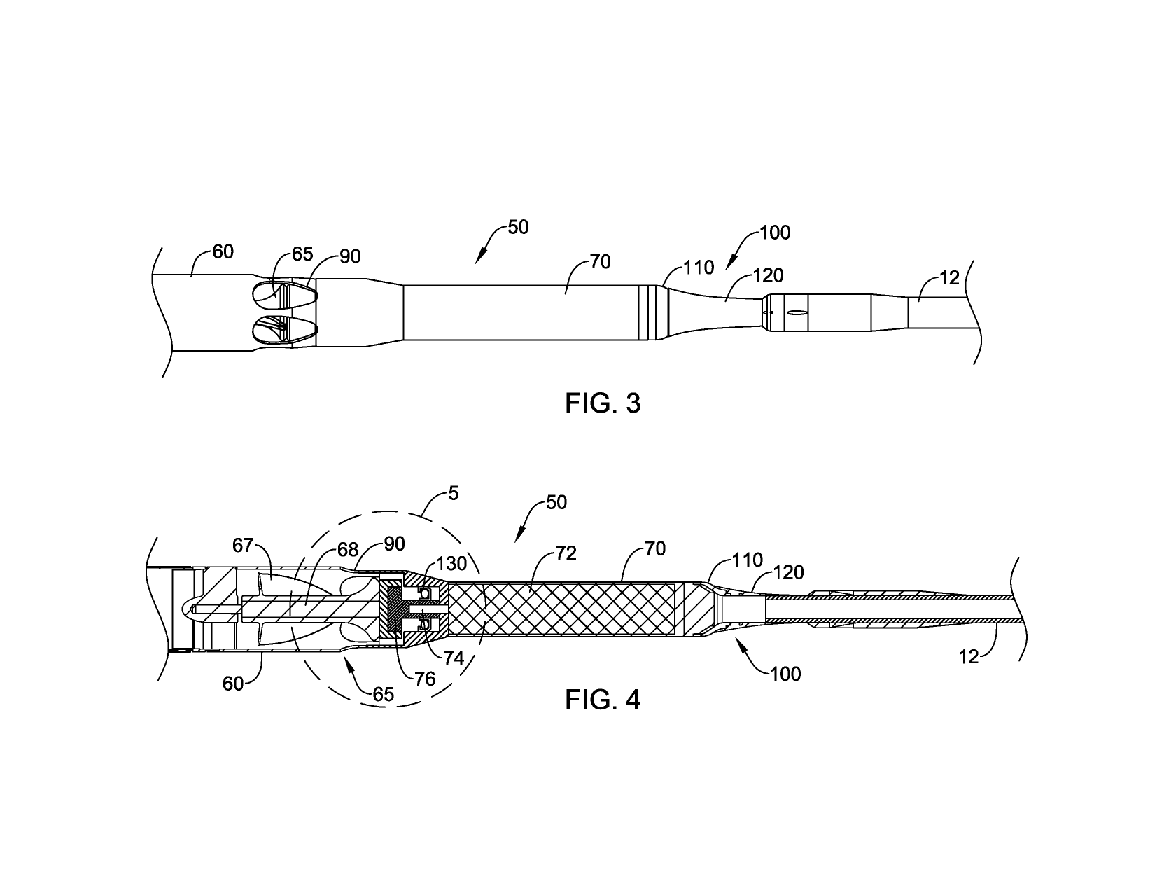

FIG. 3 is a side view of a portion of the percutaneous circulatory support device 10 illustrating a portion of the percutaneous blood pump 50 connected to the elongate shaft 12 at a junction 100. As seen in FIG. 3 and applicable to other examples of the present disclosure, an elongate shaft 12 is coupled to the junction 100. The junction 100 may include a junction end cap 110 having a proximal end region surrounding the distal end region of the elongate shaft 12, and a fillet of material 120 surrounding the proximal end region of the end cap 110 and extending proximally therefrom. The fillet of material 120 may extend proximal of the end cap 110 and surround a portion of the elongate shaft 12 extending proximal of the end cap 110.

The impeller housing 60 is positioned distal of the motor housing 70. A proximal end of the impeller housing 60 may be secured to a distal end of the motor housing 70 via a welded connection, in some instances. Applicable to this and other examples, the motor housing 70 may be connected to and/or coupled with the impeller housing 60 by processes and techniques including but not limited to: welding, adhering, bonding, molding, overmolding, extrusion, male-female couplings, threaded connections, screw fitting, press fitting, interference fitting, snap fitting, a 3-D printing process, or any combination or permutation of the aforementioned or the like. The impeller housing 60 carries the impeller assembly 65 therein. A proximal end of the impeller of the impeller assembly 65 is also visible through the outflow windows of the blood outlet 90.

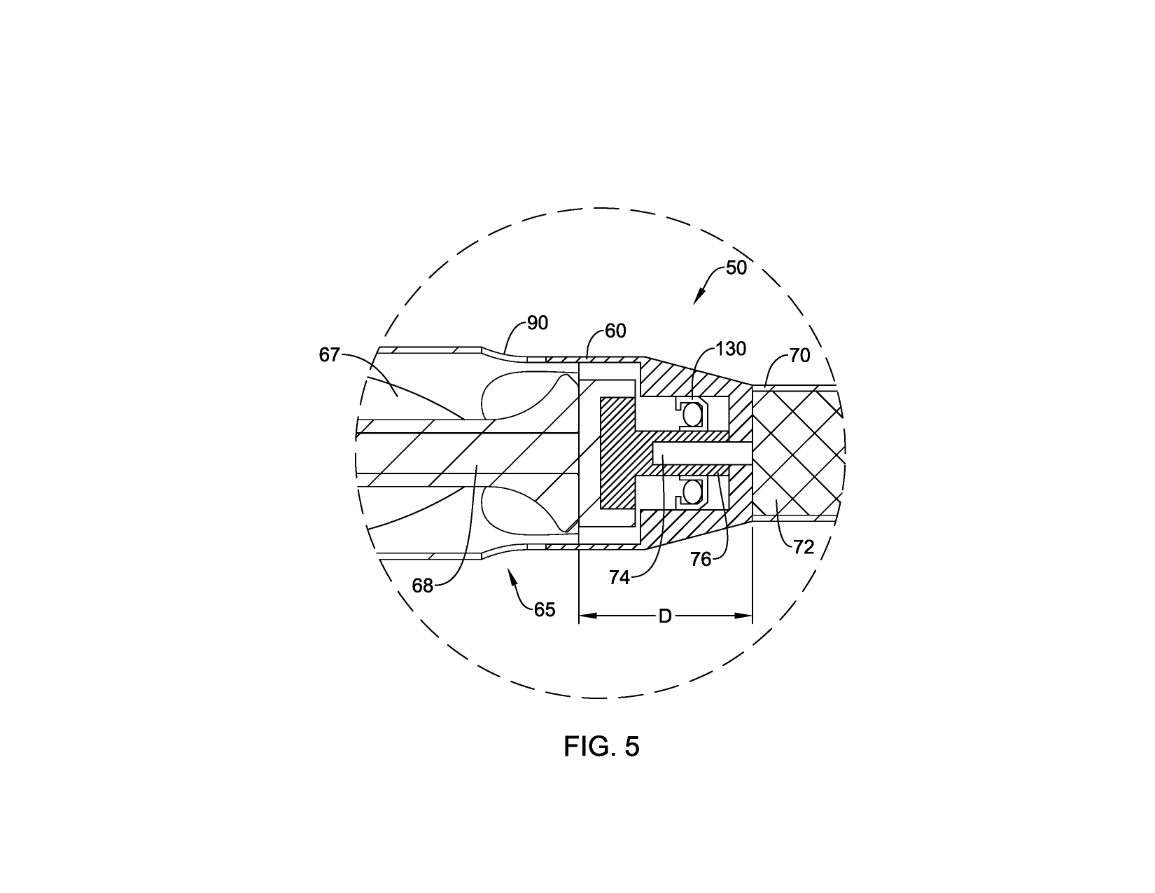

FIG. 4 is a cross-sectional view of the portion of the percutaneous circulatory support device 10 of FIG. 3. As shown in FIG. 4, the impeller housing 60 may house and/or contain the impeller assembly 65 that includes an impeller shaft 68 and an impeller 67 coupled thereto, where the impeller shaft 68 is configured and/or otherwise adapted to rotate with the impeller 67. As shown, the impeller shaft 68 is at least partially disposed within the impeller 67. As alluded to above, and in non-limiting aspects, the impeller housing 60 may partly or fully enclose and/or envelop and/or surround impeller assembly 65 and associated impeller components and features.

Continuing with FIG. 4, extending distally from the junction 100 and/or elongate shaft 12 is the motor housing 70 which houses the motor 72. The motor 72 includes a drive shaft 74 extending distally therefrom. A drive sleeve 76 may circumferentially surround the drive shaft 74, such as a distal end region of the drive shaft 74. The drive sleeve 76 may be fixed or otherwise secured to the drive shaft 74 such that rotation of the drive shaft 74 rotates the drive sleeve 76. In other words, the drive sleeve 76 may rotate with the drive shaft 74. Accordingly, through operation of the motor 72, the drive shaft 74 may be rotationally driven by the motor 72, which may also rotate the drive sleeve 76 as drive sleeve 76 is disposed upon and secured to the drive shaft 74. In examples, the drive sleeve 76 may be disposed circumferentially around the drive shaft 74. In some non-limiting examples, the drive sleeve 76 may be disposed upon the drive shaft 74 through a variety of techniques, including press fitting the drive sleeve 76 onto the drive shaft 74, through an interference fit between the drive sleeve 76 and the drive shaft 74, welding the drive sleeve 76 to the drive shaft 74, securing the drive sleeve 76 onto the drive shaft 74 through a screw fitting and/or threaded connection, and/or a snap-fit connection and/or through molding the drive sleeve 75 onto the drive shaft 74, through coating the drive sleeve 76 onto the drive shaft 74, or through other like techniques and/or processes. In yet other non-limiting examples, two or more drive sleeves 76 may be provided, and implemented in identical or similar manners as described above.

The drive sleeve 76 may be composed of a metallic material, a ceramic material, or other desired material. In some instances, the drive sleeve 76 may be composed of stainless steel and/or polished stainless steel. The circumferential surface of the drive sleeve 76 may have a surface roughness of Ra 0.8 µm or less, Ra 0.4 µm or less, Ra 0.1 µm or less, or Ra 0.05 µm or less, providing a relatively smooth surface.

As shown in FIG. 4 and in accordance with the present disclosure, the motor 72 may be positioned proximal of the impeller assembly 65 and within motor housing 70 or additionally and/or alternatively within another housing such as the impeller housing 60. In this and other examples, the impeller shaft 68 and the drive shaft 74 may be of monolithic construction and/or integral. In other words, the drive shaft 74 may directly connect and/or couple to and/or with the impeller shaft 68 to form a continuous and/or contiguous feature. In other non-limiting examples, the impeller shaft 68 may be connected to and/or otherwise coupled with the drive shaft 74 and/or the drive sleeve 76. In yet other non-limiting examples, the impeller shaft 68 may be connected to and/or otherwise coupled with the drive shaft 74 and/or the drive sleeve 76 by an interference fit, a screw fit, a snap fit, a press fit, a threaded connection, a male-female coupling and/or connection, by bonding, adhering, a 3-D printing process, or any other feasible process and/or technique known in the art.

The motor 72 is shown within the motor housing 70. Distal of the motor 72 is a seal assembly 130, although in other non-limiting examples, the seal assembly 130 may be positioned in any directional relation and/or proximity and/or at any feasible distance to the motor 72. The seal assembly 130 may form a fluid seal between blood within the impeller housing 60 and the motor 72 within the motor housing 70. Thus, the motor 72 may be sealingly isolated from any blood flowing through the impeller housing 60, while the distal end region of the drive shaft 74 and/or the distal end region of the drive sleeve 76 (which extend distally beyond the seal assembly 130) may extend into the impeller housing 60 and be exposed to (in contact with) blood flowing through the impeller housing 60. Seal assembly 130 may be fixed relative to the impeller housing 60 by any feasible technique and/or process including but not limited to: adhesion, welding, bonding, molding, press-fitting, screw-fitting, interference-fitting, a 3-D printing process, an intermediate coupling and/or connection, or other feasible techniques and/or processes known in the art.

The seal assembly 130 may be provided within the impeller housing 60 with a seal housing of the seal assembly 130 fixed relative to an inner wall of the impeller housing 60. The seal assembly 130 may include a lip seal, gland seal, and/or lip seal mechanism and/or gland seal mechanism as will be described further herein. The seal assembly 130 may interact and/or contact the drive sleeve 76 in order to promote and provide sealing engagement with the drive sleeve 76 and prevent fluid (e.g., blood) from entering the motor housing 70 and/or the motor 72, thereby removing the need for a flushing conduit or other known means for removing, transporting, displacing and/or diluting fluid (e.g., blood) that may contact a motor assembly in prior art blood pump devices. Therefore, satisfying many unmet needs in the art.

Seal assembly 130 may be a lip seal, a gland seal, a spring-loaded lip seal, a spring-loaded gland seal, a self-energizing spring-loaded lip seal, a self-energizing spring-loaded gland seal or any appropriate sealing mechanism and/or construction known in the art. It should be noted that a self-energizing spring-loaded seal is distinguished from other seals by including at least one spring member that both stores potential energy and discharges kinetic energy due to the construction and material characteristics of the at least one spring member. In other words, a self-energizing spring-loaded seal will have one or more spring members that are constructed and arranged in a fashion such that the one or more spring members store potential energy (i.e., latent and stored tensile and/or compressive and/or radially directed force within the one or more spring members) and discharges kinetic energy (i.e., the transformation and dispersal of stored tensile and/or compressive and/or radially directed force from the one or more spring members).

Providing a seal assembly such as a lip seal, gland seal, or the equivalent within seal assembly 130 eliminates the need for the use of motor purge fluid and further disallows blood from entering the motor housing 70, in other words preventing ingress of blood into the internal components and other components of the motor 72 so as to prevent motor failure and other damage to the device and associated components. It can be appreciated that this aforementioned incorporation also removes the need for any fluid flushing conduit or other similar structure intended to deliver fluid or other like substances through the motor housing 70, since the motor housing 70 is completely sealed (i.e., a fluid and/or air-tight seal) such that blood and/or other substances do not enter the motor housing 70 and therefore do not exert damage upon the motor housing 70 and associated components enclosed, as they are therefore also sealed within the motor housing 70.

In some non-limiting examples, the seal assembly 130 may include an elastomeric and/or otherwise flexible, substantially flexible, and/or at least partially flexible sealing member, which will be described further herein. The seal assembly 130 may include one or more sealing members, and the one or more sealing members may include one or more elastomeric and/or flexible and/or substantially and/or partially flexible features which aid in achieving sealing engagement between the drive sleeve 76 and the seal assembly 130 such that fluid (e.g., blood) is prevented from entering the motor housing 70 as well as the motor 72.

In some non-limiting examples, the seal assembly 130 may include one or more spring members. The one or more spring members may be one or more tension springs, one or more compression springs, one or more leaf springs, one or more helical springs, one or more springs having at least one helical profile (e.g., having a helically shaped cross-section and/or feature) or any combination or permutation of the aforementioned.

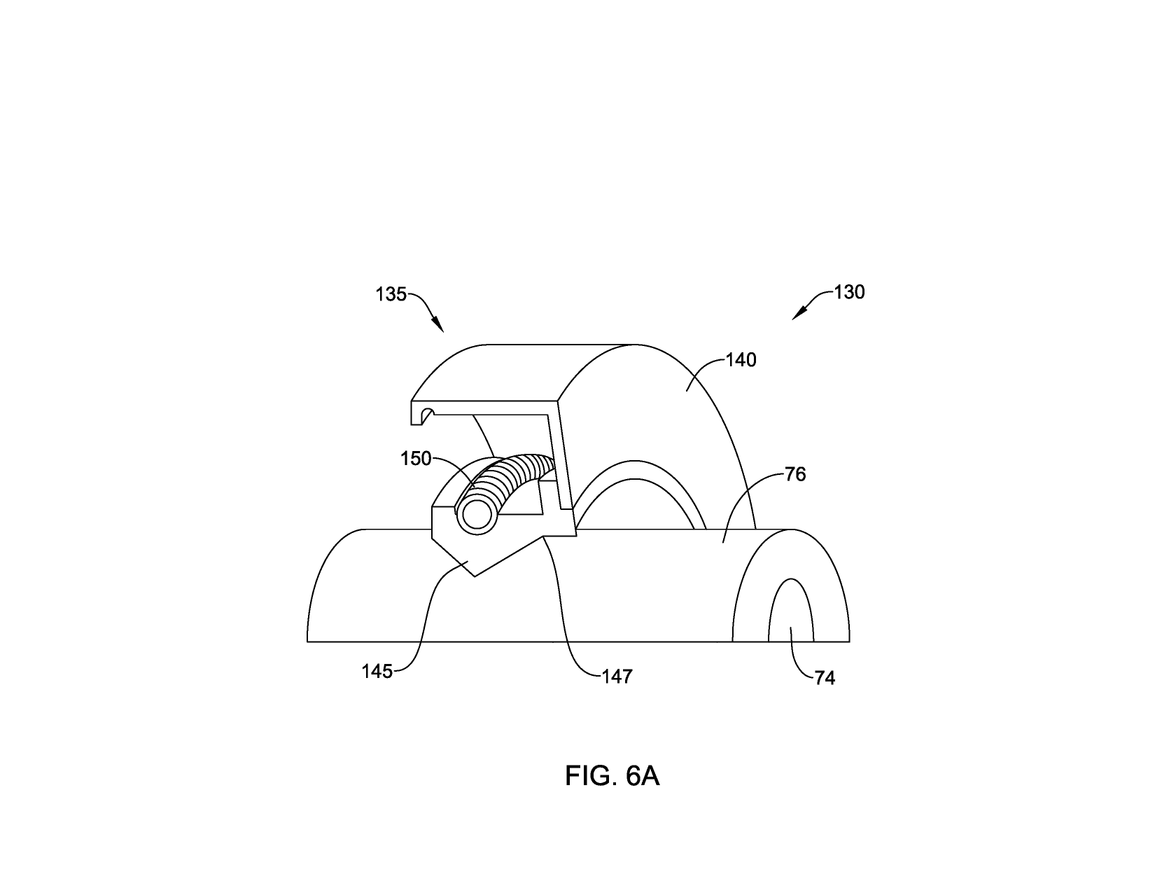

Turning to FIG. 5, an enlarged view of a portion of FIG. 4, identified within the dashed circular line 5, shows a lip-seal drive mechanism. The seal assembly 130 engages with the drive sleeve 76 (e.g., circumferentially surrounds and engages a circumferential surface of the drive sleeve 76) in fluid-tight sealing engagement such that no fluid may enter the motor housing 70 and therefore the motor 72. The drive sleeve 76 may be disposed circumferentially and/or concentrically around a part, a portion, a distal end, a distal end region, an intermediate region, or on the entirety of the drive shaft 74 which is operably connected to and/or otherwise coupled to the motor 72. The impeller housing 60 may extend distally of the motor housing 70 and/or motor 72, with the drive shaft 74 and/or the drive sleeve 76 extending into the impeller housing 60. The drive sleeve 76 may be secured to the impeller 67 such that the impeller 67 and the drive sleeve 76 rotate together via rotation of the drive shaft 74 of the motor 72. In some instances, the drive sleeve 76 may include an enlarged diameter distal end region configured to mate with, couple to, or otherwise engage an enlarged proximal end region of the impeller 67. For example, the proximal end region of the impeller 67 may surround the enlarged diameter distal end region of the drive sleeve 76 in some instances. The distal end region of the drive sleeve 76 may have a first diameter and the proximal end region of the drive sleeve 76 may have a second diameter different from the first diameter. The first diameter may be greater than or less than the second diameter. The seal assembly 130 (e.g., lip seal) may circumferentially surround and contact the proximal end region of the drive sleeve 76 proximal of the distal end region of the drive sleeve 76. Due to the direct connection between the drive shaft 74 and the impeller 67, the motor 72 may be moved closer to the impeller 67, thereby reducing the overall length of the rigid portion (including the motor housing 70 and the impeller housing 60) of the blood pump 50. For example, a distance D from a distal end of the motor 72 to a proximal end of the impeller 67 may be 10 mm (0.394 inches) or less, 8 mm (0.315 inches) or less, 5 mm (0.197 inches) or less, in some instances.

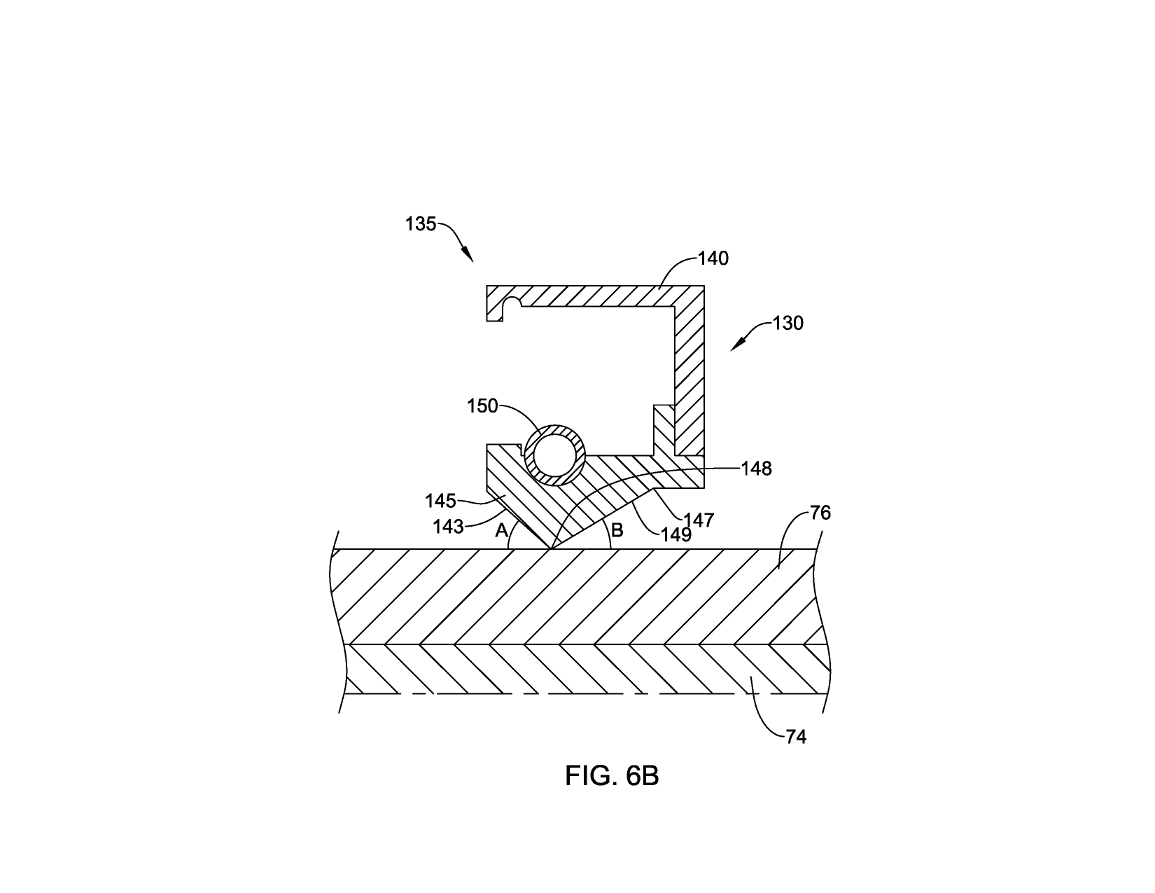

Turning to FIG. 6A, an enlarged view of an example seal assembly 130 is shown. In this and other examples, the seal assembly 130 may include a shell 140 or seal housing and a sealing member 145 coupled thereto. The shell 140 may include a radially outward facing surface 135 configured to be secured to a radially inward facing surface of the impeller housing 60 such that the shell 140 is fixed relative to the impeller housing 60. The seal assembly 130 may also include a spring member 150, in some instances referred to as a garter spring. The spring member 150 may be a tension spring, a compression spring, a leaf spring, a helical spring, a combination of the aforementioned, or other material possessing a spring constant such that the spring member 150 stores potential energy and releases kinetic energy in a radially inward direction. In other words, the spring member 150 may store potential energy and release kinetic energy in a radially inward direction toward the drive shaft 74 and/or the drive sleeve 76 and/or other components that reside within the radius of an arc and/or circle and/or substantially arcuate and/or substantially circular shape formed by the spring member 150 (e.g., a shape or form that encircles at least a part of the drive shaft 74 and/or the drive sleeve 76 or other component within the encircling form or shape).

The shell 140 may be operably connected to, adhered to, attached to, and/or coupled with the sealing member 145. In coordination with the spring member 150, the sealing member 145 may be urged against a surface of the drive sleeve 76 (e.g., a circumferential surface of the drive sleeve 76). In other words, the spring member 150 may be configured and/or otherwise adapted to apply a force to the sealing member 145 to urge the sealing member 145 and/or compress the sealing member 145 into sealing engagement (i.e., forming a fluid and/or air-tight seal) with one or more surfaces of the drive sleeve 76, such as a circumferential surface of the drive sleeve 76.

The sealing member 145 may be made of an elastomeric material, a polymeric material, a low-friction material, a lubricous material and/or an otherwise flexible material. In some non-limiting examples, the sealing member 145 may be coated and/or impregnated with a lubricious material and/or substance and/or a combination of lubricious materials and/or substances. In some non-limiting examples, the sealing member 145 may be coated and/or impregnated with an anti-coagulant substance, such as heparin, to prevent blood coagulation on the sealing member 145. In some non-limiting examples, the sealing member 145 may include one or more sealing members, two or more sealing members, or three or more sealing members located along different parts and/or portions of drive sleeve 76 and/or incorporated within a single seal assembly 130 or multiple seal assemblies.

The sealing member 145 may also include a hinge 147. The hinge 147 may be made integral with the sealing member 145, or may be a separate part and/or a component adhered to, connected to, and/or otherwise coupled with the sealing member 145. In this and other examples, the hinge 147 may be a living hinge. In other words, the hinge 147 may be a living hinge as it may be made monolithically with the sealing member 145 and/or made integral with the sealing member 145, but may be designed with different geometric, structural, and/or material constraints, such as a differing thickness, dimension, orientation, angle, topography, microscopic surface features, nanoscopic surface features, lubricity, diameter, flexibility, rigidity, pliability, torsional characteristics, and/or tensile characteristics to that of the sealing member 145. As shown in FIG. 6A, the hinge 147 may be a concavity and/or convergence of angled surfaces formed within, upon and/or integral with the sealing member 145. In other non-limiting examples, the hinge 147 may be formed as a notch, a groove, a furrow, a depression, and/or other similar structure or relief pattern. In yet other non-limiting examples, the hinge 147 may be formed as one or more notches, one or more grooves, one or more furrows, one or more depressions, and/or a combination and/or permutation of the aforementioned structures and features.

It can be appreciated that hinge 147, and the plurality of hinges described herein perform myriad effective functions in coordination with the sealing member 145 that include, but are not limited to: improving sealing engagement with the drive sleeve 76 by allowing greater flexibility within the sealing member 145, improving the efficacy and durability of the sealing member 145 by increasing the conformity of the sealing member 145 about and/or around the drive sleeve 76 (thereby at least reducing the cyclical structural fatigue of the sealing member 145), in addition to other known, effective and inherent functions of a hinge and/or hinge member.

FIG. 6B shows a schematic drawing of an example seal assembly 130 in accordance with the present disclosure. In addition to similarly indicated elements of FIG. 6A, example non-limiting aspects are shown. The seal member 145 may include a contact point 148, such as a sealing lip, sealing engaging the circumferential surface of the drive sleeve 76. In some instances, the contact point 148 may be an apex of converging surfaces of the seal member 145, as shown in FIG. 6B. In other instances, the contact point 148 may be a flat and/or convex surface contacting the circumferential surface of the drive sleeve 76. The contact point 148 may have an axial length of contact with the circumferential surface of the drive sleeve 76 of about 0.2 mm (0.0079 inches) to about 0.5 mm (0.0197 inches), or about 0.3 mm (0.0118 inches), for example.

In some instances, the drive sleeve 76 may include an annular groove extending around the circumferential surface of the drive sleeve 76 at the contact point 148 such that the apex of the seal member 145 extends into, contacts, or otherwise is engaged with the annular groove. In other instances, the drive sleeve 76 may include an annular rim extending around the circumferential surface of the drive sleeve 76 at the contact point 148 such that the apex of the seal member 145 extends over, contacts, or otherwise is engaged with the annular rim.

In the embodiment of FIG. 6, the sealing member 145 includes a distally facing tapered surface 143 facing and/or configured to be in contact with blood within the impeller housing 60 and a proximally facing tapered surface 149. The proximally facing tapered surface 149 may taper towards and converge with the distally facing tapered surface 143 at the contact point 148. The distally facing tapered surface 143 may be oriented at an acute angle A to the rotational longitudinal axis of the drive sleeve 76 and drive shaft 74. The proximally facing tapered surface 149 may be oriented at an acute angle B to the rotational longitudinal axis of the drive sleeve 76 and drive shaft 74. The angle A may be greater than the angle B to enhance the sealing characteristic of the sealing member 145. In some instances the angle A may be in the range of 35° to 50°, 40° to 50°, or about 45°, while the angle B may be 40° or less, 30° or less, 25° or less, or 20° or less, for example. The spring member 150 may be longitudinally aligned with and radially outward of the contact point 148 or sealing lip, thereby exerting a radially inward force on the contact point 148 or sealing lip to urge the contact point 148 or sealing lip against the circumferential surface of the drive sleeve 76. The hinge 147 may be positioned proximal of the contact point 148 or sealing lip and the spring member 150 to provide a desired flexibility of the seal member 145.

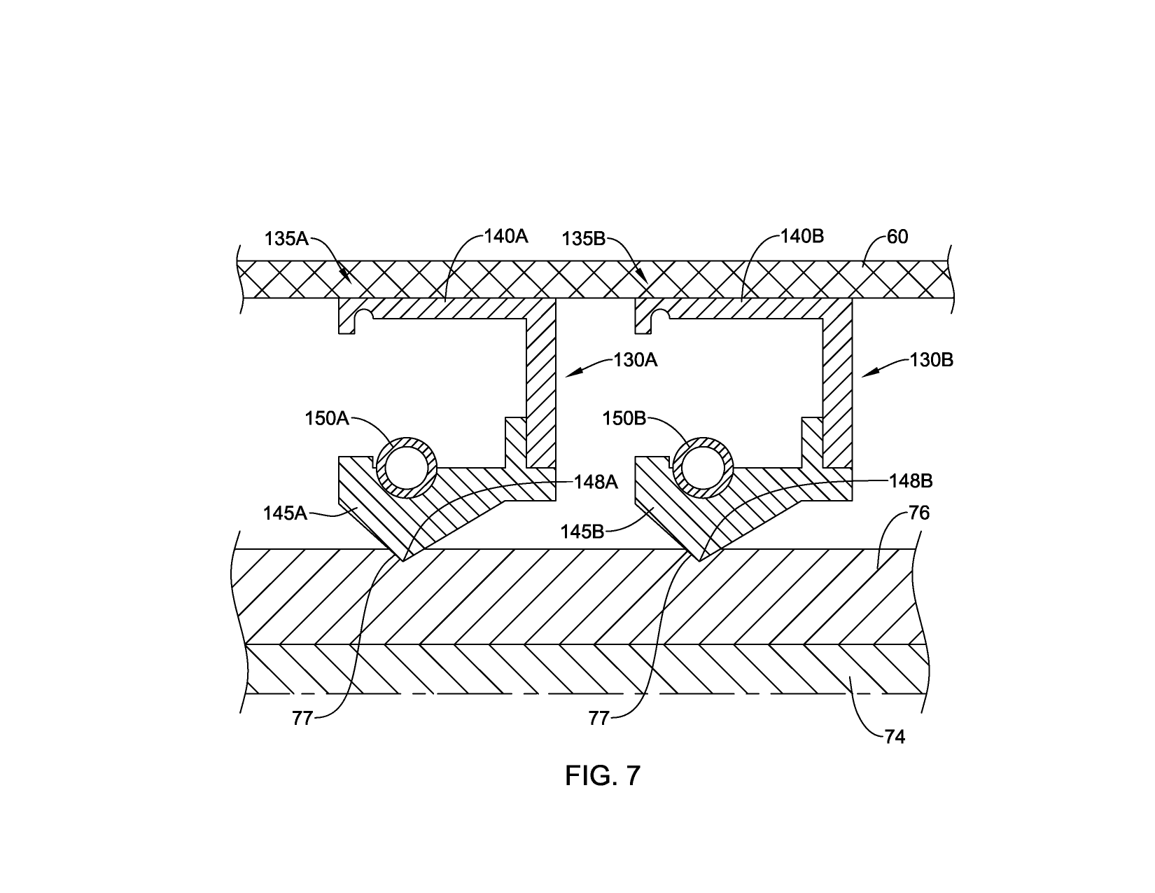

In some instances, the seal member 145 may include a plurality of lips contacting the circumferential surface of the drive sleeve 76 and/or the seal assembly 130 may include a plurality of seal members 145. Each of the plurality of lips of the seal member 145 and/or each of the plurality of seal members 145 may be spaced apart from one another, enhancing the sealing interface with the circumferential surface of the drive sleeve 76. For example, FIG. 7 is a cross-sectional view showing an embodiment including a first seal assembly 130A and a second seal assembly 130B surrounding the drive sleeve 76 and drive shaft 74, and longitudinally spaced apart from one another. The first seal assembly 130A may include a first seal housing 140A fixed to an interior wall of the impeller housing 60 and/or the second seal assembly 130B may include a second seal housing 140A fixed to an interior wall of the impeller housing 60. In some instances the first seal housing 140A may be a separate component from the second seal housing 140B, however, in other instances, the first seal housing 140A and the second seal housing 140B may be combined as a single monolithic structure. The first seal assembly 130A may include a first seal member 145A contacting the circumferential surface of the drive sleeve 76 and the second seal assembly 130B may include a second seal member 145B contacting the circumferential surface of the drive sleeve 76. For example, the contact point 148A of the first seal member 145A may engage or contact the circumferential surface of the drive sleeve 76 at a first location on the drive sleeve 76 and the contact point 148B of the second seal member 145B may engage or contact the circumferential surface of the drive sleeve 76 at a second location on the drive sleeve 76, spaced apart from the first location. Thus, the contact points 148A and 148B may be spaced apart from one another. The first seal assembly 130A may also include a spring member 150A and/or the second seal assembly 130B may also include a spring member 150B. The spring members 150A, 150B may be longitudinally aligned with and radially outward of the contact point 148A, 148B of the respective seal member 145A, 145B, thereby exerting a radially inward force on the contact point 148A, 148B to urge the contact point 148A, 148B against the circumferential surface of the drive sleeve 76.

Also shown in FIG. 7, but equally applicable to other embodiments of the disclosure, including FIG. 6B, the drive sleeve 76 may include an annular groove 77 extending around the circumferential surface of the drive sleeve 76 at the contact point 148 of the first seal assembly 130A and/or the second seal assembly 130B such that the apex of the seal member 145A, 145B extends into, contacts, or otherwise is engaged with the respective annular groove 77.

The seal assembly 130, which may be referred to as a lip seal, prevents ingress of blood proximally past the seal assembly 130 into the internal components and other components within the impeller housing 60 and/or the motor housing 70 proximal of the seal assembly 130. Thus, the seal assembly 130 may allow for the direct drive of the impeller assembly 65 by the motor 72 while preventing motor failure and other damage to the device and associated components within the motor housing 70.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous blood pump, comprising:
an impeller housing;
an impeller assembly disposed within the impeller housing; the impeller assembly including an impeller rotatably positioned within the impeller housing;
a motor positioned proximal of the impeller assembly;
a drive shaft rotationally driven by the motor;
a drive sleeve disposed circumferentially around at least a portion of the drive shaft;
a seal assembly comprising a seal housing and a sealing member secured to the seal housing;
wherein the seal assembly is fixed relative to the impeller housing; and
wherein the sealing member contacts a circumferential surface of the drive sleeve in sealing engagement therewith such that the drive shaft is rotatable with the impeller.

2. The percutaneous blood pump of claim 1, wherein the seal assembly includes a spring member configured to apply a force to the sealing member to urge the sealing member against the circumferential surface of the drive sleeve.

3. The percutaneous blood pump of claim 2, wherein sealing engagement between the sealing member and the drive sleeve is maintained by the spring member as the drive shaft rotates.

4. The percutaneous blood pump of claim 1, wherein the drive sleeve is composed of stainless steel and/or polished stainless steel.

5. The percutaneous blood pump of claim 4, wherein the drive sleeve has a surface roughness of Ra 0.8 µm or less.

6. The percutaneous blood pump of claim 1, wherein the drive shaft extends distally from the motor into the impeller housing.

7. The percutaneous blood pump of claim 1, wherein the drive sleeve is fixed to the impeller assembly and rotatable therewith.

8. The percutaneous blood pump of claim 1, wherein the drive sleeve is fixed to a distal end region of the drive shaft.

9. The percutaneous blood pump of claim 1, wherein a distance from a distal end of the motor to a proximal end of the impeller is 10 mm or less.

10. The percutaneous blood pump of claim 1, wherein the lip seal is a spring-loaded, self-energizing lip seal.

11. A percutaneous blood pump, comprising:
an impeller housing;
an impeller assembly disposed within the impeller housing, the impeller assembly including an impeller rotatably positioned within the impeller housing;
a motor positioned proximal of the impeller assembly and including a drive shaft extending distally therefrom, the motor configured to rotationally drive the impeller assembly;
a drive sleeve circumferentially surrounding and affixed to a distal end portion of the drive shaft;
a seal assembly comprising a seal housing and a sealing member secured to the seal housing;
wherein the sealing member contacts a circumferential surface of the drive sleeve in sealing engagement therewith such that the drive sleeve is rotatable therein with rotation of the impeller.

12. The percutaneous blood pump of claim 11, wherein the seal assembly includes a spring member configured to apply a force to the sealing member to urge the sealing member against the circumferential surface of the drive sleeve.

13. The percutaneous blood pump of claim 11, wherein the drive sleeve has a surface roughness of Ra 0.8 µm or less.

14. The percutaneous blood pump of claim 11, wherein sealing engagement between the sealing member and the drive sleeve is maintained as the drive shaft rotates.

15. The percutaneous blood pump of claim 11, wherein the drive sleeve is fixed to a distal end region of the drive shaft.

16. The percutaneous blood pump of claim 11, wherein a distance from a distal end of the motor to a proximal end of the impeller is 10 mm or less.

17. A percutaneous blood pump, comprising:
an impeller housing;
a motor housing positioned proximal of the impeller housing;
an impeller assembly disposed within the impeller housing; the impeller assembly including an impeller rotatably positioned within the impeller housing;
a motor disposed within the motor housing;
a drive shaft rotationally driven by the motor;
a drive sleeve circumferentially surrounding a distal end region of the drive shaft and affixed thereto;
wherein the drive sleeve is operably coupled to the impeller assembly such that the drive sleeve rotates with the drive shaft and the impeller;
a seal assembly disposed within the impeller housing, the seal assembly comprising a lip seal circumferentially surrounding the drive sleeve and forming a fluid seal therebetween;
wherein the lip seal provides a fluid seal between the impeller housing and the motor housing.

18. The percutaneous blood pump of claim 17, wherein the seal assembly includes a spring member configured to apply a force to the sealing member to urge the sealing member against the circumferential surface of the drive sleeve.

19. The percutaneous blood pump of claim 18, wherein the drive sleeve includes a distal end region having a first diameter and a proximal end region having a second diameter less than the first diameter.

20. The percutaneous blood pump of claim 19, wherein the lip seal surrounds and contacts the proximal end region of the drive sleeve proximal of the distal end region.
